(19) United States
(12) Patent Application Publication
(10) Pub. No.: US 2017/0065521 A1
(43) Pub. Date: Mar. 9, 2017

(54) BIOPHARMA APPLICATION OF MICELL TECHNOLOGY

(71) Applicant: Micell Technologies, Inc., Durham, NC (US)

(72) Inventor: Wenda C. Carlyle, Prairie Farm, WI (US)

(73) Assignee: Micell Technologies, Inc., Durham, NC (US)

(21) Appl. No.: 15/260,579

(22) Filed: Sep. 9, 2016

Related U.S. Application Data
(60) Provisional application No. 62/215,998, filed on Sep. 9, 2015.

(51) Int. Cl. A61K 9/14; A61K 9/48; A61K 9/20; A61K 9/00; A61K 9/50

(52) U.S. Cl. CPC A61K 9/14; A61K 9/0019; A61K 9/0053; A61K 9/50; A61K 9/2077; A61K 9/4808

(57) ABSTRACT

Drug delivery systems are disclosed which include a drug in dry powder form and a biodegradable or metabolizable carrier having an average particle size of less than about 1 mm for delivery of the drug to a particular location in the body and for providing for the timed elution of the drug at that location, preferably by exhibiting a linear drug elution profile for a sustained drug release period of at least 30 days. Methods for manufacturing these drug delivery systems are also disclosed.

### CROSS-REFERENCE TO RELATED APPLICATIONS

This application claims the benefit of the filing date of U.S. Provisional Patent Application No. 62/215,998 filed Sep. 9, 2015, the disclosure of which is hereby incorporated herein by reference.

### BACKGROUND OF THE INVENTION

A recent development which is particularly applicable to the preparation of drug eluting stents has been made by Micell Technologies, Inc. This development employs as a critical step in the application of drugs and polymers to such stents a supercritical fluid/electrostatic deposition technique which permits these components to be deposited in a dry powder form for the solvent-free deposition of crystalline or semi-crystalline drugs. This technique, which is shown, for example, in U.S. Pat. Nos. 6,749,902, 6,756,084, 6,780,475, 8,758,428, 8,298,565, and 8,900,651 (the disclosures of which are all incorporated herein by reference thereto), is one in which a system known as RESS (rapid expansion of supercritical solutions) is preferably employed, and involves dissolution of a polymer into a compressed gas, typically a supercritical or near-critical fluid, followed by rapid expansion into a chamber at lower pressure, typically near atmospheric conditions. The rapid expansion of the compressed gas through a small opening, with its accompanying decrease in density, reduces the dissolution capacity of the fluid, and results in the nucleation and growth of polymer particles. The atmosphere of the chamber is an isolated “cloud” or gas in the chamber. Carbon dioxide, hydrocarbon, hydrofluorocarbon, or other appropriate gas is employed to prevent an electrical charge from being transferred from the substrate to the surrounding environment. This technology has been further applied to the application of drugs, such as rapamycin, to drug eluting stents. Thus, where conventional processes for spray coating stents require drug and polymer to be dissolved in solvents, by using this improved technique solvents are no longer required, and the drug and/or polymer can be provided in a dry powder form. In some cases, the drug material can be applied to the stent in a dry powder form either concurrently or sequentially with the compressed gas application of polymer. Thus, compressed fluids are employed in this solvent-free deposition methodology. This technique thus allows for processing at lower temperatures and the absence of liquid-phase solvents to preserve the qualities of the active agent and the polymer matrix itself, as well as the ability to incorporate multiple drugs while minimizing deleterious effects from direct interactions between them, and/or their excipients, as well as a dry deposition, enhanced adhesion and mechanical properties of various layers applied to the stent's framework. In addition, this technique is particularly advantageous in that it provides for the preparation of drug products which do not include an initial drug “burst,” can be used to apply the drug in the form of microparticles and even nanoparticles, it permits the drug to be loaded onto absorbable matrices, it permits a high drug loading of up to about 40% of the API, and therefore can accommodate macromolecular drugs, it can provide for long term drug delivery (up to about 9 months, depending on the drug), and significantly it provides a linear pharmaceutical profile.

The inventors of the present application have now discovered ways to apply this technology to various additional drug delivery systems.

### BRIEF SUMMARY OF THE INVENTION

In accordance with the present invention, this and other advantages have now been realized by the discovery a drug delivery system comprising a drug in a dry powder form and a biodegradable or metabolizable carrier having an average particle size of less than about 1 mm for delivering the drug to a preselected location in the body of a patient, and providing for the timed elution of the drug at the predetermined location. Preferably, the drug is at least partially crystalline. By employing the present invention it has been unexpectedly discovered that a drug delivery system can be employed which not only provides a linear drug release profile, but also provides for sustained drug release over an unexpectedly long period of time, such as for up to 30 days, preferably up to 60 or 90 days, and most preferably up to 120 days or more. No such results have been previously obtainable.

In accordance with this invention, the technique discussed above, using a process which applies the drug in the form of a dry powder and which maintains the drug's crystalline or semi-crystalline form, is applied to create various dosage forms of the drug. The dosage forms can now include drug/polymer depots, preferably including a biodegradable and/or metabolizable polymer, nanoparticles of the drugs, such as for use by injection, implantable drug-containing wafers, transdermal drug particles and drug formulations, and drug in the form of coatings for application to various carriers.

In accordance with one embodiment of the drug delivery system of the present invention, the drug is admixed with the carrier.

In accordance with another embodiment of the drug delivery system of the present invention, the drug is applied to the carrier in the form of a coating.

In accordance with another embodiment of the drug delivery system of the present invention, the drug delivery system comprises an implant, an intravenous composition, a coated substrate, a drug delivery depot implanted subcutaneously for systemic release, an injectable depot formulation or an orally-ingestible composition. Preferably, in the case of an IV composition, the drug delivery system comprises a plurality of microparticles, and preferably a plurality of particles having a particle size of less than about 10 μm. In another embodiment, the injectable depot formulation comprises a plurality of particles having a particle size of between about 30 and 1,000 μm. These particles can come in various forms, but are preferably spherical particles. In the case of drug delivery depots which are implanted subcutaneously for systemic release, these particles will preferably have a rod-like configuration, while these particles also preferably have a particle size of less than about 1 mm, in this case, considering their preferred rod-like shape, they will have a corresponding size determined by their shortest aspect length. Thus, for example, a rod with a shortest aspect length of 1 mm can comprise a rod-like particle with a diameter of 1 mm and a length of 5 mm, thus providing a shortest aspect length of 1 mm.

In accordance with another embodiment to the method of the present invention, the orally-ingestible composition comprises a tablet, capsule, pill, pellet, caplet, or the like.

In accordance with the present invention, the Applicants have also discovered a method of manufacturing a drug delivery system which comprises providing a biodegradable or metabolizable carrier, and applying a drug in dry powder form to the carrier, whereby the drug delivery system can be delivered to a predetermined location in the body of a patient, and thereby provide for the timed elution of the drug to the predetermined location, preferably in the form of a linear release profile, and over a period of sustained drug release of up to 30 days, preferably up to 60 days, more preferably up to 90 days, and most preferably up to 120 days or more. In a preferred embodiment, the applying step comprises delivering the drug to the carrier by means of a compressed gas. Preferably, the drug and the carrier are admixed after the delivery step. In another embodiment, however, the drug is applied to the carrier in the form of a coating.

In accordance with the present invention, the drug delivery system includes a biodegradable or metabolizable carrier whose purpose is to deliver the drug with which it is incorporated to a preselected location in the patient's body. More particularly, the carrier is unlike a stent or metabolic element coated with a drug because it does not have any structural function in and of itself, but again is only present to act as a vehicle for delivery of the drug and/or to stabilize the drug for such delivery. The structural function of stents, for example, includes the fact that they are expandable to a significant extent such that they can expand in and structurally support the patient's arterial system. Again, this is nothing like the carriers of the present invention, where they certainly do not expand or otherwise provide structure for such diverse purposes. Further distinguishing these carriers from stents and the like is their size. They preferably have an average particle size of less than about 1 mm so that they can serve the functions spelled out herein, and be used in delivery systems such as implants, orally-ingestible compositions, as pills and capsules, and in IV compositions. Indeed, in the latter contact, when used for IV delivery, these carriers will preferably have an average particle size much less than 1 mm, and preferably less than about 10 μm.

In accordance with another embodiment to the method of the present invention, the carrier is provided by delivering the carrier by means of a compressed gas. In the preferred embodiment to the method of the present invention, the drug is at least partially crystalline. In another embodiment of the method of the present invention, the drug delivery system comprises an implant, an intravenous composition, a coated substrate, and injectable depot formulation, or an orally-ingestable composition. In a preferred embodiment, the intravenous composition comprises a plurality of microparticles. Preferably, the intravenous composition comprises a plurality of particles having a particle size of less than about 10 μm. In another embodiment, the injectable depot formulation comprises a plurality of particles having a particle size of between about 10 and 1,000 μm.

In another embodiment, the orally-ingestable composition comprises a tablet, capsule, pill, or caplet.

### BRIEF DESCRIPTION OF THE DRAWINGS

FIG. 1 is a chart depicting a comparison of the formulations of the present invention with those of lower standard dosage forms.

### DETAILED DESCRIPTION

The overall objective of the present invention is to provide a drug, preferably a drug in crystalline form, in a configuration such that the drug can elute within a predetermined and preferably lengthy time period, and most preferably at a precise location in the body. By utilizing the technology discussed above, a drug in powder form is combined with a carrier which is biodegradable or metabolizable in the body, so that after use, the system is completely cleared of any potentially harmful particles. Thus, by employing the systems discussed above for applying drugs in a dry powder form, and avoiding the use of solvent spray systems and the like of the prior art, it is now possible to apply crystalline or semi-crystalline drugs to precise locations, or even systemically, in combination with a biodegradable or metabolizable carrier in various forms, such as implants, intravenous compositions, coated substrates, injectable depot formulations, and even orally-ingestable compositions.

By utilizing the present invention, and preparing drug delivery systems by using the technique disclosed herein, important and unexpected results can now be achieved. In particular, these drug delivery systems will now not only have a linear elution profile, but even more importantly they can provide for sustained drug release over extended periods of time not readily obtainable heretofore. These results include sustained release of the drugs over periods of at least about 30 days, preferably at least about 60 days, more preferably at least about 90 days and most preferably at least about 120 days. The ability to achieve these results, particularly along with a linear drug release profile, now makes it possible to focus these drugs on particular areas of the anatomy, and even with systemic application, to do so in a much more effective way than was previously possible.

Depending upon which form the drug delivery system of this invention takes, the size, shape, or configuration of the carrier can, and in most cases will necessarily, be different. For example, in connection with intravenous administration, it is clear that small particles, such as those below about 10 μm, will be needed to provide effective circulation in the bloodstream. It will thus be necessary to produce such small particles, such as microparticles or even nanoparticles, from the biodegradable or metabolizable materials which are used herein. Indeed, with the use of nanoparticles, the localization or targeting of the drugs of the present invention can be maximized. Thus, these nanoparticles will accumulate in certain tissue, such that it is therefore possible to target the drug, such as to the liver or kidney. On the other hand, with injectable depot-like formulations, larger particles, such as from 10 to about 1,000 μm will be required, possibly including rod-like architectures (which can be up to about 10 cm in length), which ensure that the material does not migrate from the selected site of administration. One example of an implantable drug depot device is shown in U.S. Pat. No. 5,660,848, the disclosure of which is incorporated herein by reference thereto.

In connection with orally-ingestible drug formulations, it is preferred that the carrier be in particular form, which can include particles on the order of about 100 μm up to about 1 mm. However, in a preferred embodiment, these carrier particles can be in the form of nanoparticles, such as those of less than about 200 nm. In that case, the drug will therefore have improved bioavailability, particularly in the case of insoluble or poorly soluble drugs. These particles, such as nanoparticles, can then be admixed with or coated by the drug itself.

In the case of drug-coated substrates, or generally larger such substrates, various types and shapes of such substrates can be employed, depending upon the particular use contemplated therewith. For example, a mesh substrate can be used and coated with the drug in accordance with the general procedure described herein. This delivery system can then be used to spread the drug over a relatively large surface are. Most preferably, a flexible mesh substrate can be used for such purposes. On the other hand, a wafer substrate can be dry coated for use in accordance with the invention. This delivery system can be used for insertion to a required cite through an incision.

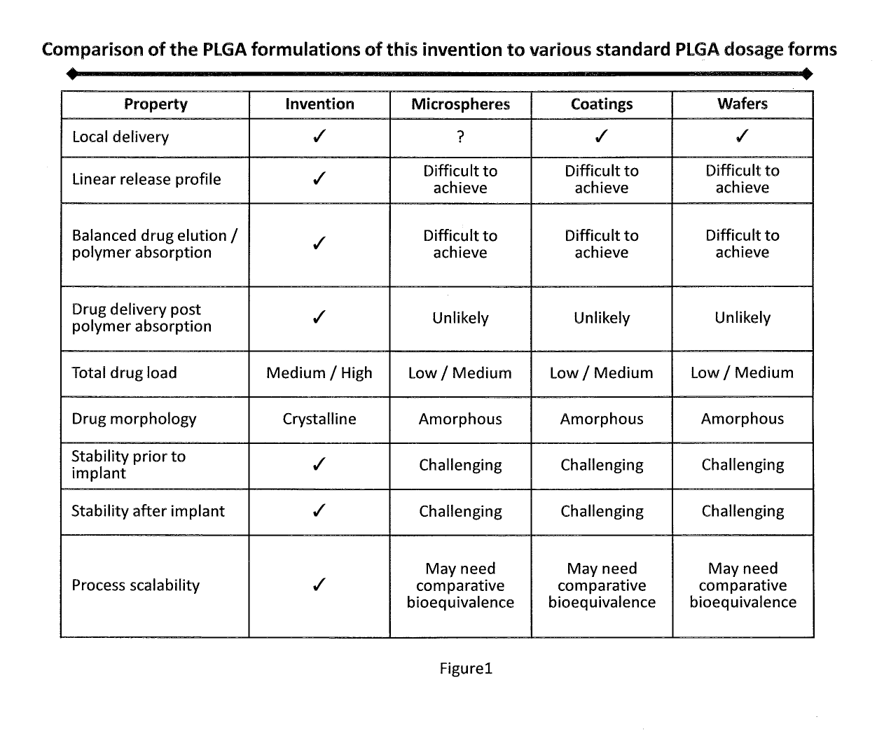

As is discussed above, the present invention preferably utilizes a carrier which is bioabsorbable or metabolizable. These include, for example, poly(lactide-co-glycolic acid) or PLGA. FIG. 1 is a chart showing a comparison of various PLGA formulations in various dosage forms, including that of the present invention.

One example of a particulate carrier or substrate which has been utilized in the past are the high molecular weight polyanhydrides of U.S. Pat. No. 4,757,128, the disclosure of which is incorporated herein by referent thereto.

With respect to the various drugs which can be utilized in connection with the present invention, one particular class of drugs are those of low bioavailability or which effect erratic first pass metabolism, and thus may have historically required intravenous infusion or parenteral injection. One example of this would be the quarternary amines, such as Pyridostigmine, which is a cholinesterase inhibitor used to treat myasthenia gravis. Because of the structure of these drugs, it is very difficult for these to become absorbed through the gut, and they have trouble crossing the blood brain barrier to reach the brain. Thus, use in accordance with the present invention, and direction of the drug to the required location of use, would be highly desirable for use in connection with these types of drugs. Another example would be the bisphosphonates. These drugs are exemplified by drugs such as Alendronate, which are used to treat osteoporosis and other bone diseases. However, the oral bioavailability of bisphosphonates is only around 0.6%. Once again, by treatment and delivery from local drug depots, an implantable, controlled, sustained release system can be used to provide long-term continuous release of the drug in the therapeutic range at the desired location.

Another such drug is Curcumin, which is an anti-inflammatory drug which, once again, has poor oral absorption. Therefore, local delivery of this drug to the sites of the inflammation in accordance with the present invention will have significant impact on the inflammation, far superior to that previously enjoyed. This drug could be delivered from an implanted drug depot, thus avoiding the problems generally associated with oral absorption. Using such an implanted depot can provide for a sustained systemic dose of the drug directly from, for example, a subcutaneous or intramuscular implant.

Another category of drugs which can find significant advantages from the present invention are highly toxic drugs, or those which have various negative side effects. Once again, in such a case these drugs are far more effective if delivered from local drug depots that can target organ specific or localized disease states without the risk of excessive and potentially deleterious systemic dosing. Thus, many of the chemotherapeutic drugs fall into this category, particularly when used to treat a localized tumor. These toxic chemotherapeutics would include drugs such as Vincristine and Actinomycin D, among many others. Also, drugs such as the alpha-reductase inhibitors for BPH and Flutamide used for treating prostate cancer, additional drugs which can have various side effects, would be highly desirable drugs to be used in connection with this invention. Thus, the number of side effects could be significantly reduced, along with the toxicity effects thereof. In this case, for example, with Flutamide and the alpha-reductase inhibitors, the dosage form could be applied by injection into the testes or prostate gland to create a local drug depot in that location. On the other hand, when these drugs are administered systemically with the delivery systems now available, there is considerable difficulty in avoiding the toxic and other side effects thereof. Even more so in this case, where these organs only obtain a small percentage of blood flow, the overall systemic doses which are necessary would need to be extremely high to achieve adequate local concentration, resulting in the appreciation of these unwanted side effects.

Another drug which could be used in connection with the present invention would be Levosimendan, which is a drug used to treat heart failure. Again, in this case, an effective oral formulation of Levosimendan has not been developed, and the drug therefore requires frequent infusion therapy. This drug can, however, be delivered by means of intracoronary administration in connection with the present invention. Long term, continuous, intracoronary administration potentially improves both drug effectiveness (improved efficiency of the beating heart) and ease of use. Similarly, the use of an intrathecal drug depot for delivery of Baclofen, a drug used for chronic pain, would be another important application of this invention. At present, intrathecal delivery requires an extensive surgical procedure involving implantation of mechanical pumps. Use of an injectable, local drug depot eliminates the morbidity associated with the surgical pump implantable procedure.

Another such drug for use in this invention would be L-DOPA, which is used for the treatment of Parkinson's disease. The very short half-life of this drug presently requires frequent dosing. On the other hand, the drug would benefit most from intracerebral delivery, but even a large stable repository, which can slowly elute the active drug from some other part of the body, would also be useful.

With respect to drugs which utilize RNAi (RNA interference), once again, a local depot of RNA could be created that targets genes important in cancer resistance, such as the multi-drug resistant gene. A local depot in the ovaries, for example, to treat ovarian cancer or a local depot in the pancreas in order to treat pancreatic cancer, would be possible with the present invention. This, therefore, would halt disease at its source by silencing the contributory genes. This is also an extremely powerful potential tool against oncogenes. In the past, failures with this drug have been linked to the rapid enzymatic degradation of the RNAi in the bloodstream. Thus, the problem can be overcome in accordance with the present invention by localizing delivery where it is needed. Also, distribution to non-target areas and immune responses thereto is a safety concern which can also be dealt with in accordance with the present invention. As another example, nano-particles of RNAi could be harnessed for encapsulating the RNAi and delivering it to the liver for treating liver-related diseases.

Turning to antibody based drugs, such as nivolumab, these can be used, for example, to treat renal cell carcinoma with immunotherapy. Nivolumab could be more effective than oral everolimus, but it presently requires infusion or injection as a mode of delivery. In accordance with the present invention, sustained release from implantable depots of these drugs, rather than frequent injections, will be rather appealing, particularly to the patient. Treatment with everolimus would be far less expensive, but oral dosage form creates less than optimum therapy. Local delivery to the kidneys from a sustained-release implanted depot of everolimus could provide more effective results compared to standard oral dosage forms.

Another application of the present invention relates to the use with statins. The corticosteroids and anti-inflammatories have generally been used as a bioactive ingredient in implanted devices. However, with the depot delivery in accordance with the present invention, there is no longer a need for an implanted device. Thus, instead of coating these drugs onto devices, such as micro screws, for example, which are permanent implants, the drug depot can be injected to hold the drug in a particular location and eluted slowly to eliminate the need for the permanent implant.

As far as an injectable drug depot which can be used in accordance with the present invention, one example would be the drug being injected into the vitrious humor of the eye, for example, or into the prostate gland. On the other hand, it is also possible to directly infuse these drug particles into the bloodstream for more general application. In either case, because of the nature of the present invention, the more linear pharmacokinetic profile which is realizable by use of the specific products in this invention allow for more precise dosing, which is important when maintenance of a therapeutic dose is normally challenged.

We have also discussed above application of the Limus drugs to the present invention. These drugs are generally limited by their poor solubility, and their low bioavailability, as well as high protein binding, variable intestinal metabolic rates, and extensive hepatic biotransformation. However, in accordance with the present invention, these drugs can now be injected into sites of inflammation, for example, such as arthritic joints. They can also be injected into tumors or implanted for drug delivery into a grafted organ, such as a transplanted kidney, to suppress local immune responses, and to possibly reduce the systemic dose of drug required. For example, with the drug rapamycin, which has a bioavailability of less than 14%, by using the present invention, the non-steady state pharmacokinetics generally obtained with drugs such as rapamycin, which requires close monitoring of serum peak and trough levels to maintain the drug in a therapeutic range, can be eliminated. For example, in the past, daily dosing has resulted in rapamycin blood concentrations ranging from 6 ng/ml to 50 ng/ml, which ranges from the non-therapeutic to the potentially toxic range. Again, this problem can be overcome in accordance with this invention. By implanting the limus drugs in a drug depot in accordance with the present invention, issues of solubility, bioavailability, and variable metabolism have been eliminated. Furthermore, these implantable drug depots provide a means to increase local effectiveness and decrease the systemic side effects of these drugs. Finally, a combination of local and systemic delivery of these immune suppressants can result in a far better therapy. Even with the limus drugs, the particular application will help determine which aspect of the present invention and which type of drug delivery vehicle is best employed. For example, for treating immune suppression, local drug depots can be delivered for adjunctive therapy for organ transplants or high-risk corneal transplants. The drug can thus be applied to the organ being implanted, possibly even in the form of an absorbable mesh, or can be delivered by an arterial implant placed in the artery feeding the new organ. For tuberous sclerosis, and the case of dermal on/near skin lesions, intracranial implant beyond the blood brain barrier can be affected. The drug can thus be injected intradermally or provided as a patch through microneedles.

In the case of cancers where mTOR contributes to accelerated tumor growth, breast, pancreatic, and renal cancers, which are currently treated with everolimus, could benefit from the present invention. Thus, drugs effective for cancers like pancreatic cancer could be injected as a local depot into the effected tissue.

In ocular applications, instead of local therapy for inflammatory eye disease and corneal transplants, in the case of the present invention, the drug can now be injected into the eye to form a local drug depot, or applied to the corneal implant prior to transplant.

In the case of neuro protection, the particular drug can be injected or implanted directly into the brain beyond the blood brain barrier. On the other hand, it could also be implanted in a method similar to the gliadel wafer used to treat malignant glioma. Thus, adjunctive therapy for multiple sclerosis, focal cerebral ischemia, traumatic brain injury, and neurodegeneration from Huntington's disease, are other potential applications.

Finally, autism and Alzheimer's disease could be treated where mTOR is a regulator of neural protein phosphorylation. Thus, potentially reducing disease progression with local and precisely controlled delivery, it would be another application in this invention.

Various modifications of the invention described herein will be apparent to those skilled in the art. Such modifications are intended to fall within the scope of the appended claims.

1. A drug delivery system comprising:
a drug in a dry powder form and a biodegradable or metabolizable carrier having an average particle size of less than about 1 mm for delivering said drug to a predetermined location in the body of a patient and providing for the timed elution of said drug at said predetermined location.

2. The drug delivery system of claim 1, having a linear drug release profile, and having a sustained drug release of at least 30 days.

3. The drug delivery system of claim 2, having a sustained drug release of at least 90 days.

4. The drug delivery system of claim 3, having a sustained drug release of at least 120 days.

5. The drug delivery system of claim 1, wherein said drug is at least partially crystalline.

6. The drug delivery system of claim 1, wherein said drug is admixed with said carrier.

7. The drug delivery system of claim 1, wherein said drug is applied to said carrier in the form of a coating.

8. The drug delivery system of claim 1, comprising an implant, an intravenous composition, a coated substrate, an injectable depot formulation, or an orally-ingestable composition.

9. The drug delivery system of claim 8, wherein said intravenous composition comprises a plurality of microparticles.

10. The drug delivery system of claim 8, wherein said intravenous composition comprises a plurality of particles having a particle size of less than about 10 μm.

11. The drug delivery system of claim 8, wherein said injectable depot formulation comprises a plurality of particles having a particle size of between about 30 and 1,000 μm.

12. The drug delivery system of claim 11, wherein said plurality of particles comprises a plurality of rod-like particles.

13. The drug delivery system of claim 8, wherein said orally-ingestable composition comprises a tablet, capsule, pill, or caplet.

14. A method for manufacturing a drug delivery system comprising:
providing a biodegradable or metabolizable carrier, and applying a drug in dry powder form to said carrier, whereby said drug delivery system can be delivered to a predetermined location in the body of a patient, and thereby provide for the timed elution of said drug to said predetermined location.

15. The method of claim 14, wherein said timed elution of said drug comprises a linear drug release profile, and has a sustained drug release of at least 30 days.

16. The method of claim 15, wherein said timed elution of said drug has a sustained drug release of at least 60 days.

17. The method of claim 16, wherein said timed elution of said drug has a sustained drug release of at least 90 days.

18. The method of claim 14, wherein said applying of said drug comprises delivering said drug to said carrier by means of a compressed gas.

19. The method of claim 18, wherein said drug and said carrier are admixed after said delivery of said drug to said carrier.

20. The method of claim 18, wherein said drug is applied to said carrier as a coating.

21. The method of claim 18, wherein said carrier is provided by delivering said carrier by means of a compressed gas.

22. The method of claim 14, wherein said carrier is provided by delivering said carrier by means of a compressed gas.

23. The method of claim 14, wherein said drug is at least partially crystalline.

24. The method of claim 14, wherein said drug delivery system comprises an implant, an intravenous composition, a coated substrate, and injectable depot formulation or an orally-ingestable composition.

25. The method of claim 24, wherein said intravenous composition comprises a plurality of microparticles.

26. The method of claim 24, wherein said intravenous composition comprises a plurality of particles having a particle size of less than about 10 μm.

27. The method of claim 24, wherein said injectable depot formulation comprises a plurality of particles having a particle size of between about 30 and 1,000 μm.

28. The method of claim 27, wherein said plurality of particles comprises a plurality of rod-like particles.

29. The method of claim 24, wherein said orally-ingestable composition comprises a tablet, capsule, pill, or caplet.
